(19) United States
(12) Patent Application Publication
(10) Pub. No.: US 2009/0318761 A1
(43) Pub. Date: Dec. 24, 2009

(54) SYSTEM AND METHOD FOR IN VIVO IMAGING

(76) Inventor: Elisha Rabinovitz, Haifa (IL)

Correspondence Address: Pearl Cohen Zedek Latzer, LLP, 1500 Broadway, 12th Floor, New York, NY, 10036, US

(21) Appl. No.: 12/377,028

(22) PCT Filed: Aug. 9, 2007
(86) PCT No.: PCT/IL07/00995

§ 371 Date: Feb. 12, 2009

Related U.S. Application Data
(60) Provisional application No. 60/836,674, filed on Aug. 10, 2006.

(51) Int. Cl. A61B 1/04

(52) U.S. Cl. 600/118

(57) ABSTRACT

An in vivo imaging system including an ingestible in vivo imaging device for obtaining images and transmitting image data; a receiver for receiving said transmitted image data; a processor for processing said image data; and a controller for controlling movement of the in vivo imaging device based on processed image data. Controlling the movement of the in vivo imaging device is typically achieved by an external magnet moved along the patient's body unconstrained by a predetermined track.

### FIELD OF THE INVENTION

The present invention relates to in-vivo imaging. More specifically the invention relates to a system and method for viewing a patient's upper gastrointestinal tract.

### BACKGROUND OF THE INVENTION

The upper gastrointestinal (GI) tract includes the esophagus and stomach. The esophagus is a muscular tubular structure about 25 cm long in adults, extending from the cricopharyngeal muscle in the pharynx to the gastroesophageal junction. Some pathologies of the upper GI tract are detailed below.

Barrett's esophagus is a premalignant metaplastic process typically involving the distal esophagus. Barrett's may develop from a condition called gastroesophageal reflux disease (GERD). Patients with GERD may develop reflux esophagitis as the esophagus is repeatedly exposed to acidic gastric contents. Over time, untreated reflux esophagitis may lead to chronic complications such as esophageal stricture or the development of Barrett's. Barrett's esophagus is diagnosed by endoscopy and histology. The line at which the columnar epithelium transitions to the squamous epithelium (i.e., the squamocolumnar junction) is known as the Z-line. Normally, the Z-line corresponds to the gastroesophageal junction. In patients with Barrett's esophagus, the columnar epithelium extends proximally up the esophagus.

Esophageal varices is a condition which is represented by dilated tortuous vessels (veins), usually submucosal, that develop due to portal hypertension (prolonged or severe). These veins often protrude into the esophageal lumen. These blood vessels may continue to dilate until they become large enough to rupture. When esophageal varices rupture, patients become acutely ill.

Endoscopy is used to examine the esophagus, stomach and the first part of the small intestine called the duodenum. Typically, detecting pathologies of the upper gastrointestinal tract includes esophagogastroduodenoscopy (EGD) with biopsy, also known as upper endoscopy. It is a procedure usually performed by a gastroenterologist (GI or intestinal doctor). This test involves passing an endoscope, a long, flexible black tube with a light and video camera on one end, through the mouth into the GI tract. This procedure involves great discomfort to the patient and may cause damage, such as perforation, to the upper GI lining.

Capsule endoscopy can be used to view a patient's entire GI tract. It involves swallowing an imaging capsule that transmits image data to an external receiver. The imaging capsule advances through the entire GI tract assisted by the natural action of peristalsis. Close inspection of a specific desired site along the GI tract may be difficult since peristalsis may advance the capsule at an unpredictable and typically uneven rate. Methods for controlling the movement of swallowable capsules have been suggested however there exists no method or system to enable a swallowable imaging capsule to controllably view a desired location in a patient's upper GI tract.

### SUMMARY OF THE INVENTION

There is provided, in accordance with some embodiments of the present invention a method and system for imaging a desired location in a patient's esophagus, for example, the Z-line. The method according to some embodiments may include the steps of receiving image data of the patient's GI tract from a capsule endoscope, substantially in real-time and using an external controlling unit to control the movement or orientation of the capsule endoscope inside the body, based on the received image data. According to some embodiments a controlling unit need not be used. An external magnet may be controlled and manipulated by a user, such as a physician.

A system according to embodiments of the invention may include an ingestible in vivo imaging device for obtaining images of the GI tract and for transmitting image data to an external receiving system. According to some embodiments the system may include, a receiver/recorder to receive and optionally record image data transmitted from the imaging device (e.g., ingestible capsule).

According to embodiments of the invention the system further includes means for controlling the imaging device movement while it is in the upper GI tract, such as in the esophagus or in the stomach. The means for controlling the imaging device movement may include a magnetic field generator such as an array of electromagnet or a set of permanent magnets. Alternatively a single external magnet may be used. According to one embodiment the magnetic field generator includes an array of magnetic elements positioned outside the body, typically on the patient's upper part of the torso. The ingestible imaging device may include a paramagnetic metal part as part of the device housing or as a component enclosed in the device housing or attached to the device. According to one embodiment the interaction between the a magnetic field generated outside the body and the paramagnetic part inside the imaging device is calculated such that the force generated is capable of stopping the progress of the imaging device along the esophagus and/or in the stomach or other parts of the GI tract, and maneuvering it.

According to some embodiments an in vivo imaging system of the invention may include an ingestible in vivo imaging device for obtaining images and transmitting image data; a receiver for receiving said transmitted image data; a processor for processing said image data; a controller for controlling movement of the in vivo imaging device based on processed image data; and a display (such as a monitor of a work station) for displaying said image data. The processing can be based on automatic scene detection (for example, transition point detection, color parameter changes detection, differences in frequency bands detection, or shape parameter differences detection) or applying pattern recognition methods. The processor may be included in said receiver or in said work station.

According to some embodiments a method of the invention may include the steps of: obtaining image data in vivo by an ingestible in vivo imaging device; receiving the image data; processing the image data; and controlling movement of said ingestible in vivo device based on the processed image data. The processing may include detecting the position of said in vivo device. Controlling the movement may including automatically deciding on direction of movement or no movement of said in vivo device.

According to one embodiment the magnetic field generator may be situated on a conduit that may be placed or worn on the patient's body such that the generator may be moved on the conduit in relation to the patient's body. Typically the conduit may include several tracks and may be configured to enable movement of the generator on different trajectories. According to one embodiment the trajectories may be perpendicular to each other. The trajectories may be at other angles to each other.

According to one embodiment the conduit may be part of a vest worn over a patient's chest. The conduit may be configured to cover regions such as the cervical region (lower border of the cricoids cartilage to the suprasternal notch), the upper thoracic region (suprasternal notch to tracheal bifurcation), the mid-thoracic region (tracheal bifurcation to just above the gastroesophageal junction), lower thoracic and/or the abdominal region (gastroesophageal junction).

According to some embodiments an external magnet may be applied to a patient's body and moved in relation to the patient's anatomy in a trajectory that is not necessarily predetermined or defined by a conduit or track. For example, a physician may move an external magnet in relation to a patient's body based on image data obtained by the imaging device, preferably in real-time. According to some embodiments an external magnet may be moved in proximity to and in relation to a patient's body in accordance with information obtained from image data. Some embodiments require a free moving magnet in an external controlling unit, the magnet not being confined to a predetermined or set conduit or track. A free moving external magnet may be supported by a wearable article such as a vest, collar or other suitable articles.

According to some embodiments a magnetic field generator may include an array of electromagnets and a controller to differentially activate specific electromagnets in the array.

### BRIEF DESCRIPTION OF THE DRAWINGS

Embodiments of the invention are illustrated by way of example and not limitation in the figures of the accompanying drawings, in which like reference numerals indicate corresponding, analogous or similar elements, and in which:

FIG. 1 is a schematic illustration of an in-vivo imaging system according to an embodiment of the present invention;

FIG. 2, is a schematic illustration of a system to control an in vivo imaging device, in accordance with another embodiment of the present invention;

FIGS. 3A and 3B, are schematic illustrations of a system for controlling an in-vivo imaging device, in accordance with another embodiment of the present invention;

FIG. 4 is a schematic illustration of an in-vivo imaging system in association with the digestive system, in accordance with embodiments of the present invention;

FIG. 5 is a flow-chart of a method, according to one embodiment of the present invention; and

FIG. 6 is a flow chart describing a method for imaging in vivo an area of interest.

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

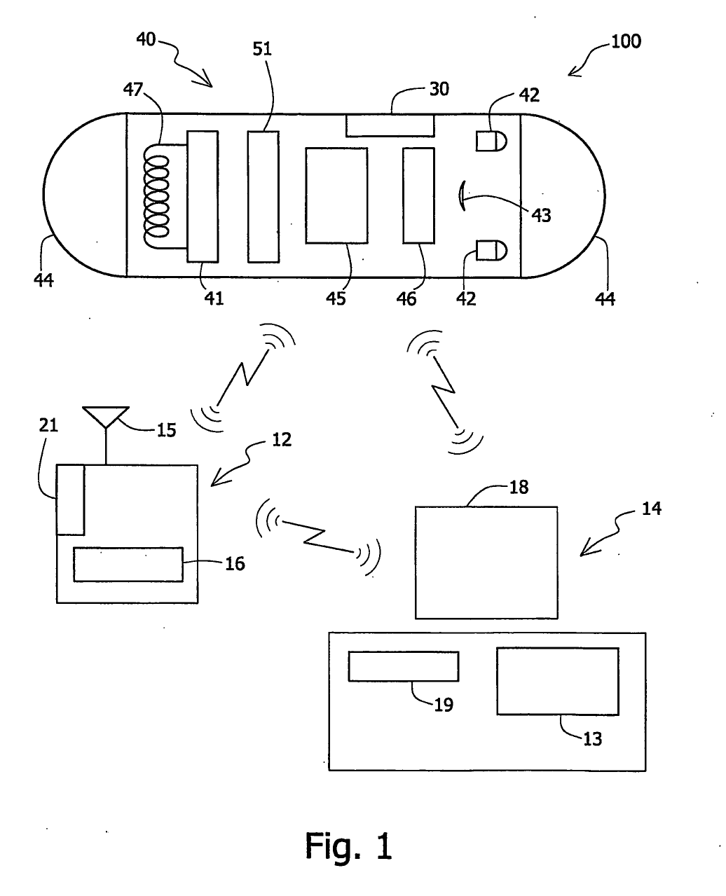

Reference is made to FIG. 1, which shows a schematic diagram of an in-vivo imaging system 100 according to one embodiment of the present invention. The in-vivo imaging system 100 may include an in-vivo imaging device 40 having, for example an imager 46, for capturing images, an optical system 43 for focusing images onto the imager, an illumination source(s) 42 such as a white LEDs (Light Emitting Diode), OLEDs (Organic LED) or other suitable illumination sources, for illuminating the body lumen. According to an embodiment of the invention the illumination source illuminates the body lumen through viewing window 44 and light rays from the body lumen are remitted to the imager through the viewing window 44. According to an embodiment of the invention the device also includes a power source 45 for powering device 40, and a transmitter/receiver 41 with antenna 47, for transmitting and/or receiving signals. Typically the transmitter transmits image data to an external device such as a receiver/recorder 12.

In some embodiments, imager 46 may include, for example, a CCD (Charge Coupled Device) camera or imager, a CMOS (Complementary Metal Oxide Semiconductor) camera or imager. Other suitable imagers, cameras, or image acquisition components may be used. According to some embodiments each frame of image data may include 256 rows, each row may include 256 pixels, and each pixel may include data for color and brightness according to known methods. According to other embodiments 320×320 pixel imager may be used. Pixel size may be between 3 to 10 micron. In another embodiment higher or lower resolution may be used. According to some embodiments pixels may be each fitted with a micro lens.

Transmitter/receiver 41 may operate using radio waves; but in some embodiments, transmitter/receiver 41 may transmit data via, for example, wire, optical fiber and/or other suitable methods. Other suitable methods or components for wired or wireless transmission may be used.

According to some embodiments the in vivo imaging device 40 may include a magnetic portion that can respond to a magnetic field that is generated outside a patient's body. The magnetic portion may be part of the device body or housing. In another embodiment imaging device 40 may include a magnetic disk or ring or other shaped magnet 51 enclosed within the device housing. The magnetic portion can be pre-magnetized in a preferred direction or passively react to external induced magnetic field. Preferably such magnet is made of a super magnet such as neodymium iron boron or another magnet made of rare earth metal or any other suitable paramagnetic material. According to some embodiments components of the device, such as the power source 45 (which may include batteries), may be used as a magnetic element.

In one embodiment, all of the components may be sealed within the device body (the body or shell may include more than one piece); for example, the imager 46, the optical system 43, the illumination sources 42, the power source 45, the transmitter/receiver 41, the antenna 47 and magnet 51, may all be sealed within the device body.

In some embodiments of the present invention, in-vivo device 40 may include one or more sensors 30 other than and/or in addition to imager 46, for example, temperature sensors, pH sensors, pressure sensors, blood sensors, etc. In some embodiments of the present invention, device 40 may be an autonomous device. According to some embodiments the device is cylindrically shaped or may have a capsule shape.

Devices according to embodiments of the present invention, including imaging, receiving, processing, storage and/or display units suitable for use with embodiments of the present invention, may be similar to embodiments described in U.S. Pat. No. 5,604,531 to Iddan et al., entitled IN-VIVO VIDEO CAMARA SYSTEM, and/or in U.S. Pat. No. 7,009,634 to Iddan et al., entitled DEVICE FOR IN VIVO IMAGING and/or in U.S. patent application Ser. No. 10/046,541 entitled A SYSTEM AND METHOD FOR WIDE FIELD IMAGING OF BODY LUMENS, all of which are assigned to the common assignee of the present invention and which are all hereby incorporated by reference.

The in-vivo imaging device 40 may, according to some embodiments of the present invention, transmit information such as in-vivo image data or other data to the receiver/recorder 12 placed or installed within the range of the transmitting distance of device 40. The receiver/recorder 12 may include an antenna or antenna array 15 and a data storage unit or memory 16. The receiver/recorder 12 may have suitable configurations and may not include an antenna or antenna array. In some embodiments of the present invention, the data receiver/recorder 12 may, for example, include processing power and/or a LCD display for displaying image data. In another embodiment receiver/recorder 12 is an integral part of the workstation 14.

According to some embodiments automatic detection of image data may occur in the receiver/recorder 12. The receiver/recorder 12 may be in communication with a means for controlling the device 40 from outside the patient's body. For example, the receiver/recorder 12 may be in communication with a controlling device that may operate the magnetic field generator to control the device 40 movement in the body by manipulating the magnetic field generated outside the patient's body, for example, based on automatic scene detection or applying pattern recognition methods typically carried out in the receiver/recorder 12.

According to some embodiments of the present invention, the receiver/recorder 12 may, for example, transfer the received data to a work station 14, which may include a computing device or personal computer, where the in-vivo image data may be further analyzed, stored, and/or displayed to a user. Typically, the image data is displayed substantially in real-time. According to some embodiments initial processing of the image data can be done in the imaging device itself or in the receiver/recorder 12 to enable real-time viewing. According to other embodiments the data is stored in receiver/recorder 12 and is then downloaded to the work station 14 for off-line viewing by a professional. Work station 14 may typically include standard components such as a processing unit 13, a memory, for example storage 19, a disk drive, a monitor 18, and input-output devices, although alternate configurations are possible. Monitor 18 may be a conventional video display, but may, in addition, be any other device capable of providing an image, a stream of images and/or other data. Instructions or software for carrying: out a method according to an embodiment of the invention may be included as part of the work station 14, for example stored in storage 19. In some embodiments, the receiver/recorder 12 may include a link 21 such as for example a USB, blue-tooth, radio frequency or infra-red link, that may connect to antenna 15 or to a device attached to antennas 15.

According to some embodiments of the present invention the memory 16 may be fixed in or removable from receiver/recorder 12. In some embodiments memory 16 may hold approximately 10 Gigabytes of memory.

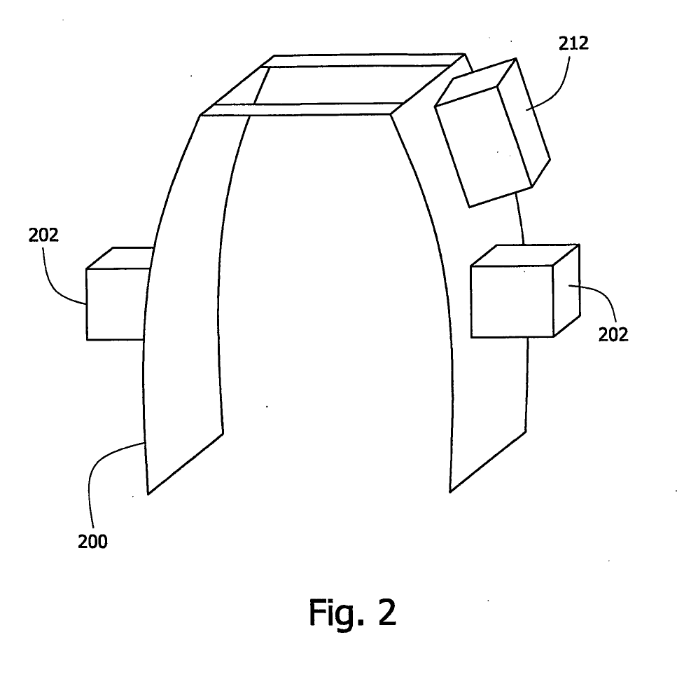

FIG. 2 shows a schematic imaging system according to embodiments of the invention. According to some embodiments the system includes a vest 200. The vest 200, which may be worn on a patient's body, for example, on the upper part of the patient's torso, includes, according to some embodiments, a magnetic field generator which includes magnets 202 for controlling the movement of an in vivo imaging device. The magnetic field generator may include electromagnets or an array of electromagnets that can be operated by a manual or automated switching board. In another embodiment magnet 202 is a permanent and/or constant magnet that may be moved along different trajectories upon the vest 200. According to some embodiments magnet 202 may be supported by vest 200 (such as by being attached by a cord to vest 200 so that the vest may carry the weight of the magnet) but may be moved in a trajectory that is not necessarily determined by a conduit.

According to some embodiments the system further includes a receiver 212. The receiver 212 may include an antenna to receive image or other data from an in vivo imaging device. Typically the device may transmit data using radio frequencies and the receiver 212 may be an RF receiver however, other transmitting/receiving methods are possible.

According to one embodiment receiver 212 may include a processor for automatic detection of predefined scenes or image data. According to other embodiments automatic detection may be carried out in a work station. According to some embodiments automatic detection may include methods such as transition point detection, detecting color parameter changes, differences in frequency bands, shape parameter differences and other appropriate methods. Based on the automatic detection magnets 202 can be directed by a controller also included in 212, e.g., the processor, to automatically control the movement of the device in vivo.

According to another embodiment receiver 212 may include a display or may be connected to a display. A physician or user may view images transmitted from an in vivo device in real-time and may, based on the images being viewed, use the magnets 202 or a magnetic field generated by an array of magnets to control the movement of the device in vivo.

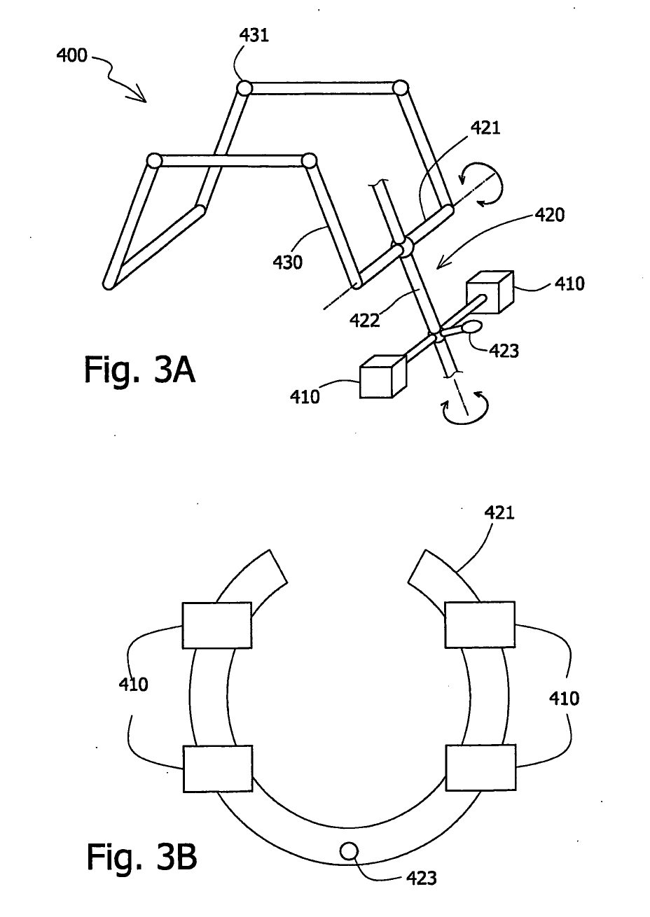

Reference is now made to FIGS. 3A and 3B which are a schematic illustration of an in-vivo imaging system in accordance with embodiments of the present invention. FIG. 3A schematically shows a system having mechanical maneuvering capabilities. According to one embodiment an external magnetic system 400 to control the in vivo device is placed on the body exterior. The external magnetic system 400 may include a set of external magnets 410 an external maneuvering system 420 capable of maneuvering the magnets 410 and a typically light weight construction 430 to support the external magnetic system 400. The external magnets 410 are capable of generating a magnetic field high enough to control the maneuverability and/or maneuver imaging device 40. A single magnet can be used however in this case the imaging device 40 may be pulled towards the single external magnet, applying pressure on the esophagus, in which case the patient may suffer discomfort associated with such pressure. According to an embodiment of the invention more than one external magnet is used such that a homogeneous magnetic field is created. A homogeneous magnetic field may enable controlling the movement of imaging device 40 with minimal discomfort to the patient and high maneuvering flexibility to the examiner.

According to one embodiment the external magnets 410 are connected to the external maneuvering system 420. The external maneuvering system 420 may contain a slide, track or rod 421 that enables sliding the external magnets horizontally and a slide, track or rod 422 that enables sliding of the external magnets vertically. The two rods 422 and 421 can be connected by a pivot or any other means that enables rotating the rods in any desirable angle to each other. According to one embodiment the external magnets 410 are connected to the maneuvering system through a pivot and handle system 423. The pivot and handle system 423 may enable tilting the external magnetic field to enable rotating and/or tilting the imaging device 40 to increase and/or improve the viewing angel that can be covered using this device.

According to some embodiments the construction 430 may support a magnet attached to it by a cord or other suitable attaching means.

The construction 430 can be made of rigid plastic, aluminum or any other material suitable for such a construction. Preferably the construction is made of non-paramagnetic material. The construction may include pivots or hinges 431 or any other arrangement that enables the adjustment of the construction to different patients having different body sizes. In addition pads and/or lining to increase the comfort and adjustment to the body shape can be used with construction 430. Another embodiment of the invention is illustrated in FIG. 3B.

According to one embodiment the system may be used in a manual procedure. According to one embodiment an examiner places and/or adjusts the system on the patient. The external magnets are locked in a position close to the upper part of the patient's body. An imaging device is administered to the patient, typically by swallowing. Images from the imaging device are transmitted and displayed. The device may be captured by the magnetic field generated by external magnets 410 and from this point the device can be maneuvered, e.g. led up and down the esophageal tube. Once an interesting spot has been discovered the handle system 423 can be rotated and/or tilted to enable better vision of the spot and/or the area of interest.

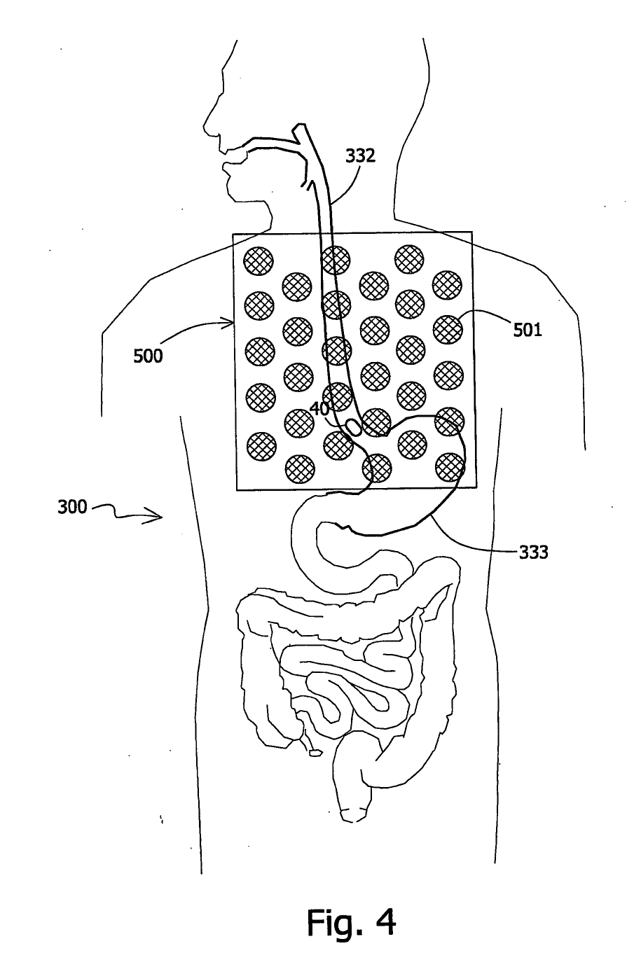

Reference is now made to FIG. 4 which is a schematic illustration of an in-vivo imaging system in association with the digestive system, in accordance with embodiments of the present invention. FIG. 4 schematically shows a swallowable imaging capsule, such as the in-vivo imaging device 40, in association with human body 300 including the esophagus 332 and stomach 333. According to one embodiment an external magnetic system 500 is placed on the body 300 either as a vest which may be worn on a patient's exterior or, according to another embodiment, with the aid of construction such as the construction 430 (for example, as described above). The external magnetic system 500 may include an array of electromagnets 501, typically coils, all connected to a central control unit (not shown). In some embodiment the array is located on the patient's front and in other embodiments the array may be located both on the front and on the back. In another example, an array of magnetic and/or electromagnetic elements may encompass and/or encircle the thorax and/or the abdominal region. A variety of other positions can be used as long as the magnetic field can be generated to capture and maneuver capsule 40. The external magnetic system 500 can be used either manually or in an automated or semiautomatic mode. During operation according to one embodiment the upper row or rows of electromagnet are activated initially. Once the imaging device 40 is administered to the patient, typically through the mouth, it is captured by the magnetic field created by two or more differentially activated electromagnets 501, and images are received and processed and possibly displayed. The capsule can be maneuvered along and/or led up and down the esophagus using a simple controller operated manually or by using a processor to automatically control and activate the electromagnets 501 to generate a magnetic 15 field so that they may move the in vivo device 40 as required. The controlling system may include a switching unit to differentially activate different electromagnets at different times to create a magnetic field through desired portions of the body and at desired angles so as to rotate and/or tilt the capsule as required. In some examples, the magnetic sensors, e.g. in the form of magnetic coils may be used to detect the position of in vivo device 40 within the magnetic field generated. Other suitable methods for detection the location and position of the in vivo device 40 may be implemented.

The in vivo device 40 as depicted in FIGS. 1 and 4 and according to one embodiment is generally capsule shaped, and may be easily swallowed and passively passed through the entire GI tract, pushed along, for example, by natural peristalsis. Nonetheless, it should be noted that the device may be of any shape and size suitable for being inserted into and passing through a body lumen or cavity, such as spherical, oval, cylindrical, etc. or other suitable shapes.

The device typically includes an imaging system for providing direct visual information of the lumen it is being propelled through. According to one embodiment the visual information can be viewed in real-time or substantially real-time and the physician viewing may control the movement of the device in the body lumen either manually using a manual system such as external magnetic system 400 or via an electronically controlled system using a joystick or similar device with an automated system e.g. external magnetic system 500. For example, the esophagus, which is a collapsed tube, in its natural state, connects to the stomach through the gastroesophageal junction. The junction is typically at an angle to the esophagus tube (His angle). Typically the His angle is 74.14+/−10.85 degrees. This angle can be significantly larger in patients with various clinical conditions. Other pathologies are found in the vicinity of the gastroesophageal junction or Z-line. According to embodiments of the invention an in vivo imaging device such as a swallowable capsule, can be controlled by the external magnetic system 400 or 500 to controllably maneuver, e.g. stop or reduce the speed (move slower) in a relevant region, such as the gastroesophageal junction. A swallowable capsule may be rotated or tilted so that the viewing window of the capsule, typically situated at one or two ends of the capsule, can optimally view an area of interest, for example, in an angled lumen.

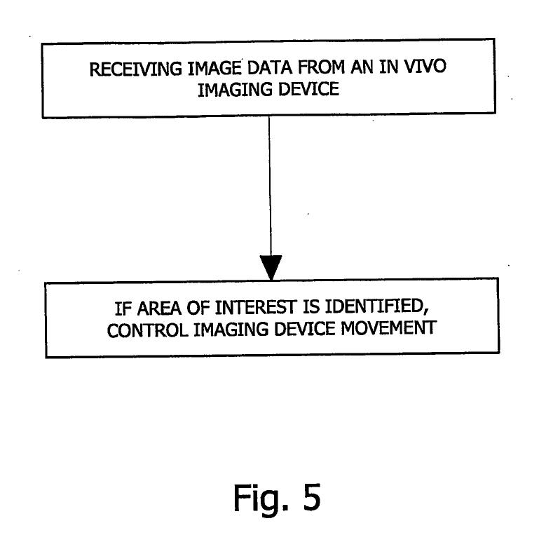

FIG. 5 is a schematic flow-chart of a method for viewing an area of interest in a patient's GI tract, for example, in a patient's esophagus. According to one embodiment the method may include the steps of, after a patient ingesting an imaging capsule, receiving image data from the imaging capsule and, based on the image data, controlling the movement or orientation of the imaging capsule to obtain optimal images of a desired location. According to one embodiment controlling the movement and/or orientation of the capsule can be done by operating a system that is located externally to the patient's body but typically in proximity to the body, to generate a force that will act on the capsule to control its progress through the lumen. According to an embodiment of the invention the system is located on the patient's torso, preferably on the upper part of the torso. According to one embodiment an array of magnets is operated outside a patient's body so as to control the movement and/or orientation of an imaging capsule. Different magnets within the array may be operated in a differential manner or in a pattern to achieve control of the capsule.

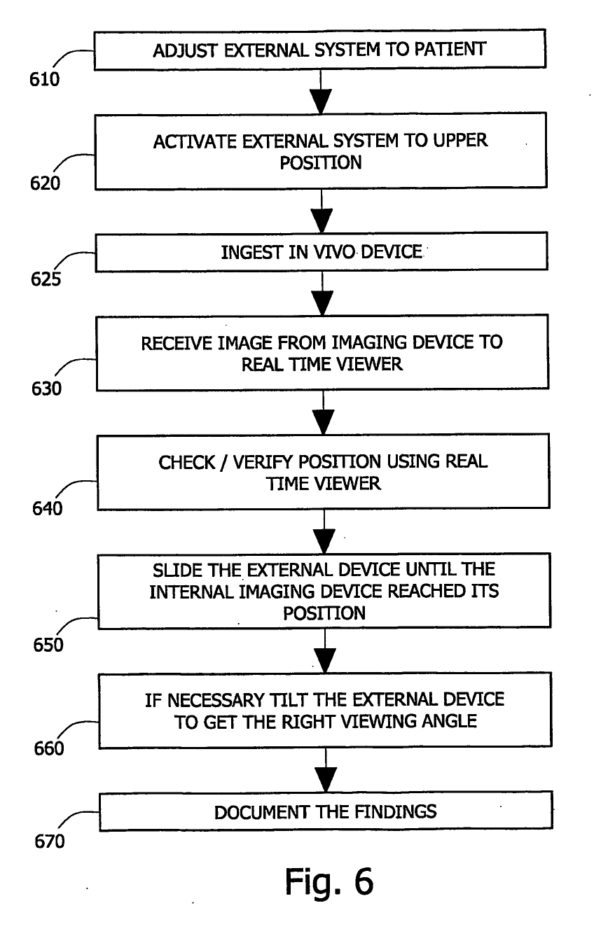

Reference is now made to FIG. 6 showing a flow chart describing a method for imaging in vivo, an area of interest. In block 610; the external magnetic system 400 and/or 500 may be positioned on the patient. The external magnetic system may be worn by the patient as a garment, e.g. a vest or may be supported by a garment or other supporting article and/or may be positioned in the vicinity of the patient by other suitable means. In block 620 the external magnetic system may be activated in the upper portion, e.g. the upper portion of the esophagus and/or the area of the esophagus closest to the pharynx. In one example, the upper portion may be activated by generating a magnetic field in the upper portion in order to catch suspend, and/or hinder the advancement of the in-vivo device before and/or in a position around an area of interest. For example, the external magnetic system may be activated in the upper portion so as to stop the in vivo device from advancing past an area of interest. According to some embodiments activating may include bringing a magnet or magnetic field generator in proximity to the required position on the patient's anatomy. In block 625, the in vivo device 40, e.g. a swallowable imaging capsule may be ingested. The in-vivo device 40, may be ingested before or after, e.g. immediately after the external magnetic system may be activated. In block 630 an image transmitted from the in vivo device may be received. The received image may be used to identify either manually or by automatic detection the position of the in vivo device within the body lumen, e.g. position of the in vivo device along the esophagus. Real-time viewing of the image frames transmitted from the body lumen may be implemented to verify, detect, and/or locating the position and/or location of the in vivo device (block 640). In other embodiments, a position tracker may be used to help determine the position of the in-vivo device, e.g. for example a magnetic sensor(s) may be used to detect the position of magnet 51 within a generated magnetic field generated using external magnetic system 400 and/or 500. In block 650, the magnetic field generated by external magnetic system 400 and/500 may be adjusted to maneuver the in vivo device to a desired position. For example the magnetic field may be adjusted to initiate forward (e.g. advancement) and or backwards (e.g. retraction) motion of the in-vivo device. In one example, a magnet or set of permanent magnets may advance, either manually by user intervention or automatically via for example a motor, to a position that will controllably advance the in vivo device to the desired position. In block 660, the magnetic field generated by external magnetic system 400 and/or 500 may be tilted and/or oriented so as to orient the in vivo device to an orientation where the imager 46 may capture a view of the area of interest, e.g. capture of view of the z-line. The in vivo device may be suspended in the desired area so that multiple image frames may be captured. Captured image frames as well as other information relating to the in vivo device may be documented and used for diagnosis (block 670). Other suitable steps and methods may be used.

According to one embodiment the method may include the steps of: bringing a magnet into proximity of a patient, for example, on or near the patient's back; inserting a capsule endoscope into the patient's GI tract, for example into the esophagus and/or stomach; viewing images obtained by the capsule endoscope; and moving the magnet in a trajectory (for example, a trajectory along a patient's back) so as to control movement of the capsule endoscope in vivo, the magnet being unconstrained by a predetermined track.

According to one embodiment received images are displayed on a work station or other monitor and based on the displayed images a user can manually manipulate a system to control an appropriate magnetic field. According to another embodiment images need not be displayed. According to some embodiments a system may be automatically or semi-automatically operated whereas a magnetic field is generated and/or manipulated based on automatic detection of images or patterns. Automatic detection may include methods such as transition point detection, detecting color parameter changes, differences in frequency bands, shape parameter differences and other appropriate methods. Based on the automatic detection permanent magnets or an array of magnets can be directed by a controller e.g, the processor, to automatically control the movement of the device in vivo.

According to embodiments of the invention an imaging capsule's passage through the esophagus can be slowed down or even completely stopped to optimally image esophageal varices. According to other embodiments a capsule's position or orientation in the esophagus may be changed, for example, tilted, to conform with the anatomy of the gastroesophageal junction to enable fill view of the Z-line or other areas of interest.

It will be appreciated by persons skilled in the art that the present invention is not limited to what has been particularly shown and described hereinabove. Alternate embodiments are contemplated which fall within the scope of the invention.

1-11. (canceled)

12. An in vivo imaging system, comprising:
an ingestible in vivo imaging device to be taken by a patient for obtaining images and transmitting image data, said device comprising a magnetic element;
an external magnetic system for controlling movement of the in vivo imaging device, said magnetic system comprising:
a construction for adjusting on the patient's body; and
an external magnet,
wherein said construction supports the external magnet, and wherein said external magnet is able to move in two perpendicular axes;
a receiver for receiving said transmitted image data;
a processor for processing said image data; and
a work station for displaying said image data.

13. The in vivo imaging system according to claim 1, wherein said external magnetic system comprises two magnets.

14. The in vivo imaging system according to claim 1, wherein said processor is processing based on automatic scene detection or applying pattern recognition methods.

15. The in vivo system according to claim 3, wherein said automatic scene detection comprises transition point detection, color parameter changes detection, differences in frequency bands detection, or shape parameter differences detection.

16. The in vivo system according to claim 1, wherein said processor is included in said receiver or in said work station.

17. The in vivo imaging system according to claim 1, wherein said ingestible in vivo device obtains images of the GI tract.

18. An in vivo imaging system, comprising:
an ingestible in vivo imaging device to be taken by a patient for obtaining in vivo images and transmitting image data, said device comprising a magnetic element; and
an external magnetic system comprising:
an array of electromagnets placed over the patient's body; and
a controller for controlling movement of the in vivo imaging device by activating different electromagnets at different times.

19. The in vivo imaging system according to claim 7 wherein said system further comprises
a receiver for receiving said transmitted image data;
a processor for processing said image data; and
a work station for displaying said image data.

20. A method comprising the steps of:
obtaining image data in vivo by an ingestible in vivo imaging device;
receiving the image data through said receiver;
processing the image data; and
controlling movement of said ingestible in vivo device based on the processed image data.

21. The method according to claim 9, wherein said processing comprises detecting the position of said in vivo device.

22. The method according to claim 9, wherein said controlling movement comprises automatically deciding on direction of movement or no movement of said in vivo device.

23. The method according to claim 9, wherein said controlling movement is done by a magnetic field generator which comprises a magnet, a set of permanent magnets or an array of electromagnets positioned outside a patient's body.

24. A method for in vivo imaging, the method comprising:
bringing a magnet into proximity of a patient;
inserting a capsule endoscope into the patient's esophagus;
viewing images obtained by the capsule endoscope;
moving the magnet in a trajectory so as to control movement of the capsule endoscope in vivo, said magnet being unconstrained by a predetermined track.

25. The method of claim 13 comprising moving the magnet along the patient's back.
